(19) United States
(12) Patent Application Publication
(10) Pub. No.: US 2009/0053294 A1
(43) Pub. Date: Feb. 26, 2009

(54) Anti Mineralocorticoid Therapy of Infection

(76) Inventor: Patrick T. Prendergast, New South Wales (AU)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(21) Appl. No.: 12/084,312

(22) PCT Filed: Oct. 31, 2006
(86) PCT No.: PCT/IE2006/000124

§ 371 Date: Jun. 3, 2008

(30) Foreign Application Priority Data
Oct. 28, 2005 (IE) 2005/0723

(51) Int. Cl. A61K 9/127; C12N 7/06; C07J 21/00; A61K 31/585; A61K 31/195; C07K 16/42; A61K 39/12; A61P 31/12

(52) U.S. Cl. 424/450; 435/238; 540/43; 514/171; 514/173; 424/131.1; 530/387.2; 424/199.1

(57) ABSTRACT

Antimineralocorticoid compounds are disclosed for use in the prophylaxis and therapy of viral infections, especially the retroviral infection by HIV. These compounds can be administered alone or in combination with conventional anti-viral agents or anti-sense mineralocorticoid Steroid ReceptorNA or DNA mutants of heat shock proteins.

The present invention is directed to therapeutic applications for Anti Mineralocorticoid compounds for their previously unappreciated, prophylactic and therapeutic antiviral properties and their other therapeutic properties disclosed herein. The invention also relates to compositions that comprise Anti Mineralocorticoid compounds and an excipient.

The acquired immune deficiency syndrome AIDS is a disease characterized by the loss of cell-mediated immunity. It is attributable to a virus that belongs to the family of lipid envelope retroviruses very prevalent in the animal kingdom and is called human immunodeficiency virus (HIV). HIV is a virus that infects and takes over certain cells of the immune system, our body's defense against infections and diseases. These cells are important in fighting disease. Once infected, the virus uses the cells to make new copies (replicates) of itself, which then go on to infect other cells. This causes the infected cells to function improperly and die prematurely, weakening the immune system.

Disturbances of the entire immunodefense mechanisms result from the HIV infection, because above all the T4 or helper cells of the T system of the specific immunodefense are prevented from carrying out their role in the regulation of the immunoresponse. The- HIV-induced T4 reduction results in the development of frequent and eventually fatal opportunistic infections caused by pathogenic organisms such as viruses, bacteria, protozoa's or fungi, normally harmless if there is a normal balance between the different T cell populations. It is thought that the immune dysregulation observed in individuals infected with HIV during progression to AIDS is accounted for by a shift from a T helper 1 (Th1) to a less protective Th2-type cytokinin profile. It is the imbalance between the Th1 and Th2 that allows diseases that would ordinarily be contained or eliminated to progress sometimes lethally. There are urgent reasons for the development of more efficacious and safer treatments of viral and retroviral infections, as the number of patients infected with HIV or AIDS virus has significantly increased in recent years.

Anti-viral agents that inhibit replication of viruses have been known since the mid 1980′s. The overall goal of anti-HIV therapy is to slow or stop the replication process, and thereby slow or stop the progression of HIV disease and the. destruction of the immune system. While other approaches of combating HIV infection have been proposed and tested, thus far only anti-viral therapy has been proven to slow HIV disease progression and extend life. Many drugs are now available for inhibiting the replication of the HIV virus, however, their side effects are often so severe that treatment must be halted allowing HIV resistant strains quickly develop. Current anti-HIV/AIDS therapies can be categorized into groups based on which step in the virus's life cycle they target or how they do it: nucleoside analogue reverse-transcriptase inhibitors; non-nucleoside analogue reverse-transcriptase inhibitors and protease inhibitors. The first two groups of therapies work by mimicking one of the building blocks of DNA, interfering with reverse transcription, a process essential for HIV to reproduce itself. The protease inhibitors, work at a later stage in the viral life cycle, after the virus as successfully infected the cell and is attempting to make new copies of itself. These drugs ultimately slow down the replication of viral DNA. However, they do not rid the body of the virus, but merely act to reduce the severity and slow the development of the infection. Ultimately it can be said that, conventional anti-HIV agents do not provide sufficient effect. Furthermore, all of the drugs used as single-agent therapy loose their effectiveness over time.

There is a need for new, less toxic and more effective treatments that work against HIV. Ideally, new therapies should rid the body of the virus rather than merely slowing it down. These new therapies would preferably be virus non-specific in their action so as to prevent the promotion of resistant strains.

Accordingly it is an object of the present invention to provide a method and composition for lowering lipid envelope viral load to undetectable levels.

Aldosterone is a mineralocorticoid, a hormone that is produced by the adrenal glands. Aldosterone acts by stimulating the Mineralocorticoid Steroid Receptor.

It is believed that aldosterone increases the reabsorption of sodium (and the excretion of potassium) by the distal tubules of the kidney. The reabsorption of sodium results in an increased reabsorption of water that can result in hypertension. Low blood aldosterone levels are seen in Addison's disease and toxemia of pregnancy. Higher levels can be seen in Cushing's syndrome, primary hyperaldosteronism, malignant hypertension, severe swelling in congestive heart failure, and nephrotic syndrome.

A number of drugs have been identified which can inhibit the activity of aldosterone in the body by blocking Mineralocorticoid Steroid Receptor, including spirolactones. The term “spirolactone” indicates that a lactone ring (i.e., a cyclic ester) is attached to another ring structure in a spiro configuration (i.e., the lactone ring shares a single carbon atom with the other ring). Spirolactones, which are coupled to steroids, are the most important class of spirolactones from a pharmaceutical perspective, so they are widely referred to in the pharmaceutical arts simply as spirolactones. As used herein, “spirolactone” refers to a molecule comprising a lactone structure coupled via a spiro configuration to a steroid structure or steroid derivative.

One particular spirolactone is called spironolactone, a synthetic steroid with an aldosterone-like structure, is classically described as an Antimineralocorticoid compound or antagonist, acting functionally as a competitive inhibitor of the Mineralocorticoid Steroid Receptor. Spironolactone belongs to a class of blood pressure lowering agents called potassium-sparing diuretics. It is used to treat essential hypertension, low potassium blood levels, and fluid retention and swelling associated with congestive heart failure, cirrhosis and other conditions in which a diuretic is needed without the associated decrease in potassium levels. It works by removing excess fluid from the body. Another use is in the diagnosis and treatment of primary hyperaldosteronism. Spironolactone is marketed as an anti-hypertensive and diuretic drug by G. D. Searle (Skokie, Ill.) under the trademarks “Aldosteronectone” and “Aldactazide.” Spironolactone is the name commonly used by chemists; the full chemical name is 17-hydroxy-7-alpha-mercapto-3oxo-17-alpha-pregn-4ene-21-carboxylic acid gamma-lactone acetate. This compound, its activities, and modes of synthesis and purification are described in a number of U.S. patents, including U.S. Pat. No. 4,529,811 (Hill and Erickson 1985). Another spironolactone effective as an Antimineralocorticoid compound is epoxymexrenone. Epoxymexrenone possesses high mineralocorticoid receptor affinity (comparable to spironolactone) but with reduced binding affinity for androgen and progesterone receptors. Initial studies of this compound have demonstrated a Na⁺ /K⁺ effect equipotent with spironolactone at a 50 mg dose. Antimineralocorticoid compounds include analogous compounds of spironolactone are exemplified potassium canrenoate [Clin. Pharm. Ther. 21, 602 (1977)], potassium canrenoate [J. Pharmacol. Exp. Ther. 209, 144 (1979)], potassium prorenoate [Clin. Pharm. Ther. 18,391 (1975)], spirorenone [Japanese Unexamined Patent Publication No. 55-1627991, dihydrospirorenone, eplerenone (U.S. Pat No. 5,981,744 and WO00033847] and the like.

The present invention also provides novel spirolactones of FORMULA 1 described in U.S. Pat. No. 4,129,564 (1978) as anti-viral agents.

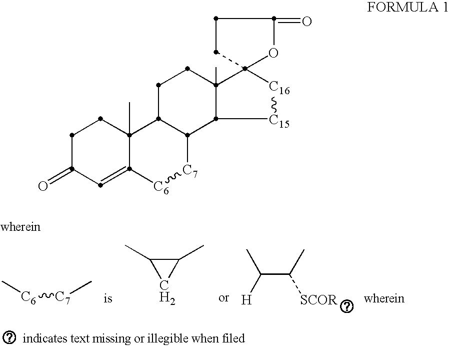

R is a lower alkyl of up to 5 carbon atoms, and

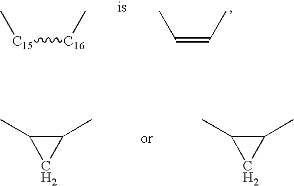

Lower alkyl residues include branched and unbranched groups, preferably methyl, ethyl and npropyl.

Spironolactone is extensively metabolised [J. Clin. Pharmacol. 29, 342 (1989)]. Canrenone is thought to be the primary metabolite, but other metabolites include: 7-alpha-thiospironolactone, 7-alpha-thiomethylspironolactone, 6-beta-hydroxy-7-alpha-thiospironolactone, and 6beta-hydroxy-7-alpha-thiomethylspironolactone.

The present invention is directed to a method of treating an individual exposed to or infected with a lipid envelope virus comprising administering to that individual a therapeutically effective amount of one or more compounds of the present invention, that inhibit, or prevent replication of said lipid envelope virus by interfering with the replicative or other essential functions of the virus, by interactively blocking the virus binding target in mammalian cells, so as to interfere in the essential life cycle activities of the virus necessary for lipid viral replication and entry to the chromosome.

The present invention relates to specific Antimineralocorticoid compounds that inhibit viral replication and display immuno-modulatory activity. It has been surprisingly found that Antimineralocorticoid compounds have potent anti-viral activity. Furthermore, the present invention relates to the metabolic derivatives (or metabolites) of specific Antimineralocortcoid compounds that also have ant-viral activity.

The inventions objects include the provision of pharmaceutical formulations of Antimineralocorticoid compounds, which are suitable for effective anti-viral therapy. Other objects are to provide methods to make and use the formulations.

In accordance with the present invention, a method is provided to treat or prevent a viral infection comprising administering to a subject an effective amount of one or more compounds of the present invention.

The present invention also provides the use of one or more of the compounds of the present invention, for the manufacture of a medicament for a viral infection.

The present invention also provides compounds of the present invention for use in a method of treatment of viral infections, said method comprising administering one or more anti mineralocorticoid compounds to a subject.

In accordance with the objects of the present invention, is provided an improved pharmaceutical formulation for use or method the prophylaxis and therapy of viral infections or a complication or consequence thereof. In particular the invention relates to the use of Antimineralocorticoid compounds and their metabolic derivatives in the prophylaxis and therapy of viral infections, viral replication and the development and prevention of the deficiency of the immune system resulting in the development of opportunistic infections and certain cancers. More especially the invention relates to the use of Antimineralocorticoid molecules and their metabolic derivatives in the prophylaxis and therapy of viral infections, an example of which is the retrovirus, thought to be responsible for the Acquired Immune Deficiency Syndrome (AIDS) and AIDS related syndromes, believed to result from infection from the Human Immunodeficiency Virus (HIV), antibodies to which are found in almost all individuals diagnosed with AIDS.

Other embodiments provide a pharmaceutical formulation or method to treat a viral infection or to ameliorate one or more symptoms associated with a viral infection such as a flaviviral or retroviral infection comprising administering to an infected patient an effective amount of a antimineralocorticoid molecule or compound or a formulation as disclosed herein.

It is an object of the present invention to provide a composition, which includes at least one Antimineralocorticoid compound for treatment, therapeutic or prophylaxis, against a viral infection. The composition containing at least one Antimineralocorticoid compound is designed so that upon administration, it has maximum bioavailability. This satisfies the need for a non-specific, antiviral treatment with immune modulatory properties.

The present invention is further directed to a pharmaceutical formulation or method in the prophylaxis and therapy against AIDS related syndromes such as cachexia and/or wasting syndrome.

Accordingly, the Antimineralocorticoid molecules and compounds disclosed herein provide a pharmaceutical formulation or a method for treating a viral infection; for use in treating any viral infection, preventing a future viral infection and/or minimizing the effects of a future infection by a virus; comprising administering to a patient in need thereof a prophylactically or therapeutically effective amount of a composition comprising at least one Antimineralocorticoid compound.

The advantage of this is that an effective anti-viral is provided that has minimal risk of generating viral resistance to antimineralocorticoid.

In another embodiment the Antimineralocorticoid compounds are delivered to viral infected cells by incorporating the Antimineralocorticoid compounds into liposomes or carbohydrate vehicles targeted to infected viral cells by placing viral directed antibodies on the surface of these liposomes or carbohydrate vesicles.

In another embodiment the Antimineralocorticoid compound is, selected from the group consisting of spironolactone, spirorenone, 1,2-dihydro-spirorenone, 1,2α-methylene-spirorenone,

7a-Acetylthio-3-oxo4, 15-androstadiene-[17(β-1′)-spiro-5′]perhydrofuran-2′-one

3-Oxo-7a-propionylthio-4,15-androstadiene-[17(β-1′)-spiro-5′]perhydrofuran-2′-one

6β,7β-Methylene-3-oxo4,15-androstadiene-[17(β-1′) spiro-51]perhydrofuran-2′-one

15a,16a-Methylene-3-oxo-7a-propionythio-4-androstene-[17(β-1′)-spiro5]perhydrofuran-2′-one

6β,7β, 15a,16a-Dimethylene-3-oxo-4androstene[17(β-1′)-spiro-5′]perhydrofuran-2′-one

7a-Acetylthio-15a, 16a-methylene-3oxo4androstene-[17(β-1′)-spiro-5′]-perhydrofuran-2′-one

7a-Acetylthio-15β,16β-methylene-3-oxo-4-androstene-[17(β-1′)-spiro-5′]-perhydrofuran-2′-one

15β,16β-Methylene-3oxo-7β-propionylthio-4-androstene-[17(β-1′)-spiro-5′]perhydrofuran-2′-one

6β,7β,15β,16β-Dimethylene-3-oxo4-androstene-[17(β-1′)-spiro-5′]perhydrofuran-2′-one
eplerenone, potassium canrenoate, canrenoate, canrenone and pharmaceutically acceptable salts thereof or their metabolites.

In another embodiment the Antimineralocorticoid compound is, selected from the group of progestogens with antimineralocorticoid activity consisting of progesterone, gestodene, drospirenone, dimethisterone, ethinyloestradiol, ethisterone, 11β-hydroxyprogesterone, 17α-hydroxyprogesterone, 16α-methyl progesterone, hydroxyprogesterone caproate, medroxyprogesterone acetate, proligestone and pharmaceutically acceptable salts thereof or their metabolites, analogues and mimic molecules.

In another embodiment the Antimineralocorticoid compound can be synthesized as a prodrug.

In another embodiment, the Antimineralocorticoid compounds is a 7α-acetylthio4-pregnene-3,20-dione represented by formula B.

In which R₁ is hydrogen, hydroxy, hydroxyl, a mineral acid ester such as sulfate, phosphate or nitrate group, or acyloxy-OR₂, the acyl group R₂ being derived from a carboxylic acid of the formula R₄OOH which may have up to 12 carbon atoms, and in which R₄ may be substituted or unsubstituted, saturated or unsaturated, straight chain or branched, alicyclic, aryl, heterocyclic or mixed and R₃ is methyl.

In one embodiment, R₁ is hydroxyl or OR₂ where R₂ is derived from a carboxylic acid of the above type, but having one or more from 3 to 12 carbon atoms.

In another embodiment R₁ is hydroxy, monocarboxylic, straight or branched-chain alkanooyloxy group having up to 12 carbon atoms.

In another embodiment R₁ is hydrogen, hydroxy, acetoxy, propionyloxy, n-butyryloxy, trimethylacetoxy, n-valeroyloxy or n-heptanoyloxy.

In another embodiment the Antimineralocorticoid compound is selected from the group comprising: 7α-acetylthio4-pregnene-3,20-dione; 7α-acetylthio-21-hydroxy-4pregnene3,20-dione; 7α-acetylthio-21-acetoxy-4pregnene-3,20dione; 7α-acetylthio-21-propionyloxy4-pregnene3,20dione; 7α-acetylthio-21-n-butyryloxy4-pregnene-3,20-done; 7α-acetylthio-21-trimetylacetoxy-4-pregnene-3,20dione; 7α-acetylthio-21-n-valeroyloxy-4-pregnene-3,20-dions; and 7α-acetylthio-21-heptanoyloxy4-pregnene-3,20-dione.

In another embodiment the Antimineralocorticoid compound is a 9,1 epoxy steroid compound, especially those of the 20-spiroxane series and their analogs.

In another embodiment, the Antimineralocorticoid compounds are halogenated in any position. The halogen can be selected from the group consisting of chlorine, bromine, fluorine and iodine.

In another embodiment the composition further includes a pharmaceutically acceptable carrier, which in one embodiment is cyclodextrin, preferably hydroxypropyl beta cyclodextrin and in another embodiment is vitamin E oil.

The advantage of administering a pharmaceutical formulation containing at least one Antimineralocorticoid compound with a suitable carrier is two fold:
1. The Antimineralocorticoid compound eliminates the virus by blocking viral-cell attachment, cellular entry and replicaition.

2. The Antimineralocorticoid compound has Immuno upregulatory properties, for example natural killer cells, and/or dendritic cells are upregulated and inflammatory cytokines are reduced.

The compounds of the invention can be formulated and administered as free bases or in the form of their pharmaceutically acceptable salts for purposes of stability, convenience of crystallization, increased solubility, and the like.

In one embodiment of the invention the infection is a viral infection, in another embodiment the viral infection is caused by a retrovirus, in another embodiment, the retroviral infection is caused by HIV or AIDS virus, Herpes virus, cytomegalovirus, or an animal virus, and In an additional embodiment the viral infection is caused by a lipid envelope virus.

In another embodiment, the pharmaceutical formulation is used to treat AIDS related syndromes, including cachexia and/or wasting syndrome or lipodystrophy.

One useful property of the Antimineralocorticoid compound and their metabolites is their anti-viral activity, which is manifested, pursuant to one aspect of the present invention, in a broad-spectrum antiviral activity. Accordingly, the present invention contemplates administering daily to a subject an amount of at least one Antimineralocorticoid compound that is clinically effective in treating or preventing a viral infection, which the subject suffers or is at risk from infection. Illustrative viruses against which the invention can be applied are HIV, cytomegalovirus (CMV), a KS-producing herpes virus, Kaposi's Sarcoma-associated herpes virus, the virus of the genus hepatitis, a virus of the genus picomaviruses, a virus of the genus molluscipoxvirus, hantaviruses, among other viruses.

Additionally, the present invention is also broadly directed at providing the use of the composition in the treatment (i.e., in the sense of treating an existing infection, preventing a future infection, minimizing the effect of a future infection) of all infections which are not retroviral infection, several representative examples of which include one or more kind of Mycoplasma, and/or one or more diseases caused by Mycoplasmas and/or one or more of the following infections: hairy leukoplakia, oral candidosis, mouth ulcerations—aphthous/herpatic/bacterial, fungal candida, squamous oral carcinoma, Kaposi's sarcoma oral lesions, periodontitis, necrotizing gingivitis, human papilloma virus, rhinovirus and arboviral molluscum contagiosum, orafacial herpes zoster, Epstein barr virus, rotaviruses, togaviruses, including alpha viruses (also known as arboviruses, group A), flaviviruses (also known as arboviruses, group B, such as yellow fever, as well as hepatitis C and hepatitis G), rubiviruses (also known as rubella viruses e.g., human rubella virus), pestiviruses (also known as mucosal disease viruses, such as bovine virus diarrhorea virus BVDV, hog cholera virus, and sheep border disease), as well as any other non-retrovira) viral induced infections. Thus virus infections that may be treated include but are not limited to HIV, SIV, FIV, FELV, SHIV, Kaposi's Sarcom-associated herpes virus and other herpes viruses (e.g. HSV-1, HSV-2, human herpes virus 6 (HHV6) and HHV-8), the viruses associated with hepatitis (HAV, HBV, hepatitis C virus [HCV]), and human cytomegalovirus, togaviruses and flaviviruses, e.g., California encephalitis virus, St. Louis encephalitis virus, western equine encephalitis virus, eastern equine encephalitis virus, Colorado tick fever virus, LaCrosse encephalitis virus, Japanese encephalitis virus, yellow fever virus, Venezuelan equine encephalitis virus, Murray valley fever virus, tick-borne encephalitis virus, GB virus A, GB virus B, GB virus C, Dengue virus 1, Dengue virus 2, Dengue virus 3, Dengue virus 4, Semliki Forest virus, Sinbis virus, picomaviruses, rhinoviruses, coronaviruses, respiratory syncytial viruses, polioviruses, parainifuenza viruses and influenza viruses ( including type A, type B, and type C. The aldosterone receptor antagonists are also useful to ameliorate one or more symptoms associated with viral infections, e.g., fever, pain or fatigue.

Pursuant to a preferred embodiment of the invention, an effective amount of Antimineralocorticoid compounds thus administered is such as to produce a circulating concentration of the Antimineralocorticoid compounds and their metabolites sufficient to reduce viral loads as monitored by, e.g., viral titer methods or by PCR.

Treatment according to the present invention can be effected when the subject is a neonate. Administration is carried out prior to delivery of the neonate and/or during delivery of the neonate.

The Antimineralocorticoid compounds according to the present invention can be administered to a patient in any of a wide range of routes. Thus, with regard to the types of formulations in which the active compounds according to the present invention can be administered, as well as any additives can be included with the active compounds in the formulations, and the possible routes of administration, it is well known to those of skill in the art that such formulations can be provided in a wide variety of types, and it is within the skill of the ordinary artisans to select a specific formulation and route of administration and then test suitability for use. By way of example but not limitation, suitable routes Include enteric, parenteral, topical, oral, rectal, nasal or vaginal routes. Parenteral routes include subcutaneous, intramuscular, intravenous, intraperitoneal, intradermal and sublingual administration. Also, compositions may be implanted into a patient or injected using a drug delivery system.

The pharmaceutical formulation according to the present invention may be administered locally or systemically. By systemic administration means any mode or route of administration that result in effective amounts of active ingredient appearing in the blood or at a site remote from the route of administration of the active ingredient.

Further, the pharmaceutical formulation according to the present invention may be administered intermittently. The advantage of this is that it allows the patient to suspend therapy for periods without the worry of inactivity of the drug resulting from the development of a resistant strain of virus.

The pharmaceutical formulation according to the invention may be formulated for enteral, parenteral or topical administration. Indeed all three types of formulations may be used simultaneously to achieve systemic administration of the active ingredient.

In one embodiment of the invention, the Antimineralocorticoid compound is micronized. In accordance with the present invention, the expression “micronized” means that the compound has been micronized in accordance with any process for micronizing, a number of which are known in the art. The micronized particles preferably include a percentage of particles, which are of a diameter, which is about 10 microns, or less, preferably 5 microns or less. For example, in a preferred aspect of the invention, at least 80% of the particles in a formulation of micronized particles have a diameter of less than 5 microns. An alternative to micronizing compound is to solubilize the compound and put it into liposomes of appropriate size. The manufacture of liposomes and the insertion of active ingredients into such liposomes are well known in the art.

For oral administration, the Antimineralocorticoid anti-viral compounds of this Patent can be formulated into solid or liquid preparations. Suitable formulations for oral administration include hard or soft gelatin capsules, dragees, pills, tablets, including soft-coated tablets, troches, lozenges, melts, powders, micronized particles, non-micronized particles, solutions, emulsions, elixirs, suspensions, syrups or inhalations and controlled release forms thereof.

One of the preferred formulations of the present invention is that the compound is enterically coated and is administered orally. In another embodiment, the compound is administered sub-lingually.

In one embodiment of the invention the enteric coating is made of a polymer, preferably selected from the group consisting of poly(lactic-glycolic acid) polyester, cellulose acetate pththalate, hydroxypropyl-methyl cellulose phthalate poly(butyl methacrylate), (2-dimethy) aminoethyl) methacrylate, and methyl methacrylate.

Solid dosage forms in addition to those formulated for oral administration include rectal suppositories.

According to a further aspect of the invention the compound is formulated in a liposome.

In another embodiment of the invention, a liposome's or cyclodextrim molecular cage are provided carrying the Antimineralocorticoid compounds targeted to HIV infected cells by putting antibodies to the HIV coat protein gp160 or gp41 on its surface. The advantage of this is that the liposome can selectively target HIV infected cells.

In another embodiment of the present invention, liposomes are provided containing high concentrations of at least one Mineralocorticoid Steroid Receptor blocker to infected cells this would preferrentially bind the aldosterone binding domain of the aldosterone receptor and prevent HIV viral protein attachment and transport into or infection off the cell. In another embodiment, the Antimineralocorticoid compound is spironolactone, or Drospirenone or a combination of these molecules.

Suitable injectable solutions include intravenous, subcutaneous and intramuscular injectable solutions. Examples of injectable forms include solutions, suspensions and emulsions. Typically the compound(s) is injected in association with a pharmaceutical carrier such as normal saline, Ringers solution, dextrose solution and other aqueous carriers known in the art. Appropriate non-aqueous carriers may also be used and examples include cyclodextrin, preferably hydroxypropyl beta cyclodextrin, mixed oils (vitamin E oil), polyethylene glycol and ethyl oleate. A preferred carrier is cyclodextrin in water. Frequently, it is desirable to include additives in the carrier such as buffers and preservatives or other substances to enhance isotonicity and chemical stability.

The Antimineralocorticoid compounds can also be administered topically. Suitable formulations for topical administration include creams, gels, jellies, mucliages, pastes and ointments. The compounds may be formulated for transdermal administration, for example in the form of transdermal patches so as to achieve systemic administration.

The composition may also be administered in the form of an implant.

The composition may also be administered in the form of an infusion solution or as a nasal inhalation or spray.

In another embodiment it may also be preferable to co-administer with at least one other anti-viral agent Examples of anti-virals include, but are not limited to nucleoside analogues (AZT; ddC; ddl; d4T; 3TC; BW 1592; PMEA/bis-POM PMEA; dOTC; DAPD); non-nucleoside reverse transcriptase inhibitors (delavirdine; DMP 266; HBY097; loviride; nevirapine, emivirine; AG1549; PNU142721; Calanolide A; DPC961); protease inhibitors (ABT-378; ritonavir; nelfinavir; BW 141; KNI-272; indinavir; saquinavir; L-756,423; DMP-450; BMS-232630); ALX40-4C; hydroxyurea; lobucavir; pentafuside; T-1249; PRO 542; FP-21399; AMD 3100; HE-2000 and peptide T.

Further examples of anti-virals include, but are not limited to Abacavir; Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Coviracil; Cytarabine Hydrochloride; Delavirdine Mesylate; Desciclovir; Didanosine; Disoxaril; Edoxudine; Emivirine; Emtricitabine; Enviradene; Enviroxime; Epivir; Famciclovir; Famotine Hydrochloride; Fiacitabine; Fialuridine; Fosarilate; Foscamet Sodium; Fosfonet Sodium; Ganciclovir, Ganciclovir Sodium; Idoxuridine; Indinavir; Kethoxal; Lamivudine; Lobucavir, Lodenosine; Lopinavir, Memotine Hydrochloride; Methisazone; Nelfinavir; Nevirapine; Penciclovir; Pirodavir; Ribavirin; Rimantadine Hydrochloride; Saquinavir Mesylate; Ritonavir; Somantadine Hydrochloride; Sorivudine; Statolon; Stavudine; Tenofovir, Tllorone Hydrochloride; Trifluridine; Valacyclovir Hydrochloride; Vidarabine; Vidarabine Phosphate; Vidarabine Sodium Phosphate; Tipranavir, Viroxime; Zalcitabine; Zidovudine; Zinviroxime.

Recent studies (Am J Physiol Endocrinol Metab 2000, 279 (2) E386-E394, The MINERALOCORTICOID STEROID RECEPTOR mediates aldosterone-induced differentiation of T371 cells into brown adipocytes, Penfornis P., viengchareun S., Le Menuet D., Cluzeud F., Zennaro M C and Lombes M) have shown that when aldosterone is applied to T371 cells they differentiate into brown adipocytes that accumulate intracytoplasmic lipid droplets, an effect accompanied by a dose dependent increase in intracellular triglyceride content. Weight gain is a very common side effect following administration of protease inhibitors to HIV patients. This results from the accumulation of adipose tissue in the trunk, neck and face producing the classic characteristic description of truncal obesity, moon face and buffalo hump. This more colloquially is referred to as crix belly. Antimineralocorticoid compounds prevent the cellular adipose accumulation. Glucocorticoid receptors were shown to have no involvement in cellular adipose accumulation because it prevents viral proteins present in protease inhibitor from acting on adipocytes as a mimic molecule of aldosterone. The co administration of Antimineralocorticoid compounds such as Spirolactone and/or Drospirenone with protease inhibitors would enhance the anti-viral effect of both classes of compounds and minimise the side effects of protease inhibitor, the current drugs of choice for treatment of HIV.

The components of any of the pharmaceutical formulations disclosed herein can be administered simultaneously (in a combination formulation), essentially simultaneously (e.g., administration of each compound a few minutes or a few hours apart), or can be administered sequentially, e.g., several days apart, or more than a week apart. For example, a compound of the present invention, at least one Antimineralocorticoid compound can be administered together, or essentially simultaneously, e.g., administration of each compound a few minutes or a few hours apart, or can be administered sequentially, e.g., several days apart, or more than a week apart. All such variations in administration of the combination therapy are encompassed within the scope of the invention.

In another embodiment, the composition is incorporated in a pharmaceutically acceptable carrier, diluents, vehicles and the like for systemic administration by feeding. An example of such a carrier is cyclodextrin.

With regard to dosage and duration of treatment according to any aspect of the present invention, it is recognized that the ability of an artisan skilled in pharmaceutical administration of drugs to determine suitable dosages depending on many inter-related factors is well known, and skilled artisans are readily able to monitor patients to determine whether treatment should be started, continued, discontinued or resumed at any given time. For example, dosages of the compounds are suitably determined depending on the individual cases taking symptoms, age and sex of the subject and the like into consideration. The amount of the compound to be incorporated into the pharmaceutical composition of the invention varies with dosage route, solubility of the compound, administration route, administration scheme and the like. An effective amount for a particular patient may vary depending on factors such as the condition being treated, the overall health of the patient and the method, route and dose of administration. The clinician using parameters known in the art makes determination of the appropriate dose. Generally, the dose begins with an amount somewhat less than the optimum dose and it is increased by small increments thereafter until the desired or optimum effect is achieved. Suitable dosages can be determined by further taking into account relevant disclosure in the known art.

In general, the amount of compound delivered to the patient is sufficient to achieve a plasma concentration of from about 3 to 10 μg/ml to about 5000 μg/ml of plasma, typically about 3 to about 50 μg/ml or about 5 to about 25 μg/ml. However, when liposomes targeted to viral infected cells are used to administer the Antimineralocorticoid compounds, high doses of 25 mg/ml are used. The effective amount is optionally administered In a dosage ranging between 10 μg/kg and about 20,000 μg/kg of body weight of the patient. Unit dosages for any of the conditions described in the disclosure will typically comprise about 1-1000 mg/day of an Antimineralocorticoid compound often about 5 to 500 mg/day, ideally 400 mg/day with an optimal blood plasma concentration of 60 μM in the blood all day. Preferable pediatric doses range from 0.001 to 100 mg/kg/day, with optimal doses in the range of 3 mg/kg/day.

Administration of the Antimineralocorticoid compounds has a direct anti-viral effect, as reported herein. This anti-viral effect is established by virtue of the ability of the Antimineralocorticoid compounds and their active metabolites to block viral cellular entry and cellular infection, as indicated in the accompanying tables and graphs.

The Mineralocorticoid Steroid Receptor like most steroid hormone receptors, mediates its biological response by aldosterone crossing the plasma membranes of cells and interacting with Aldosterone Receptor proteins in the cytosol or nucleus, to form complexes. Mineralocorticoid Steroid Receptor has been shown to be a heterooligomeric complex that includes a 90 kDa heat shock protein (HSP90).

The aldosterone/Mineralocorticoid Steroid Receptor complexes are then chaperoned by heat shock proteins to the nucleus where they then accumulate in the nucleus of cells where they bind to specific regulatory DNA sequences. The viral genetic element is translocated, and multiple copies are made, and then translated resulting in the formation of enzymes and the viral protein coat. Virus multiplication is often lethal to the cells in which it occurs and in many cases the infected cell breaks open and thereby allows the progeny viruses access to nearby cells. Many of the clinical manifestations of viral infections reflect this cytolytic effect of the virus. Both the cold sores caused by herpes simplex virus and the lesions caused by smallpox, for example, reflect the killing of the epithelial cells in a local area of the skin.

In a recent publication Mineralocorticoid Steroid Receptor have been shown to be functionally present on lymphocytes (Leukemia (2000) 14, 1097-1104, Demonstration of the mineralocorticoid receptor hormone and action in human leukemic cell lines N Mirshahi, S Mirshahi, N Golestaneh, Z Mishal, C Nicolas, C Hecquet and M K Agrwal).

The HIV viral coat proteins in this Patent have been demonstrated to act to mimic the aldosterone molecule. It is proposed that by binding the Mineralocorticoid Steroid Receptor on white blood cells, the virus readily gains access to the cellular DNA. Antimineralocorticoid compounds act by blocking the HIV viral protein targeted to the aldosterone binding domain of the Mineralocorticoid Steroid Receptor from attaching requires either the presence of aldosterone antagonists or the anti-idiotypic receptor antibody. By blocking the Mineralocorticoid Steroid Receptor it will be possible to inhibit or prevent viral replication and prevent viral incorporation into the cellular genome of the host cell.

Specific Amino-Acid sequences identified herein as viral vaccine candidate antigens are sequences on the (Mineralocorticoid Steroid Receptor) which allow for steroid binding e.g. aldosterone are identified which if presented to the uncoated entering virus or if added to the assembly of de-novo virus peptides would cause the viral protein receptor capable of binding the hydrophobic sequences bound by aldosterone to be neutralised this would inhibit viral genes being transported to the host cells nucleus.

Other Antimineralocorticoid compounds (aldosterone antagonist and/or molecules which mimic aldosterone structure) in particular compete with the viral coat proteins involved in Mineralocorticoid Steroid Receptor attachment and reduce infection rate.

In another embodiment the DNA nucleotide sequence coding for the Hydrophobic Amino Acid sequence corresponding to the aldosterone attachment site is selected from the following 15 mer consensus sequences:

| | |
|---|---|
| GRE (+) | GGTACAnnnTGTTCT; |
| GRE (−) | ATYACNnnnTGATCW. |

This above-described mechanism is utilised by a large number of virus families and the necessity to bind to the Mineralocorticoid Steroid Receptor at the aldosterone attachment site could be used to interfere with productive infection in new drug and vaccine designed using this information.

The receptor that binds the HSP CD91 is a low density lipoprotein like receptor. It has been shown that early after the exposure of permissive CD4⁺ T-lymphocyte culture to HIV-1, it is noted that a nuclear translocation of HSP70 followed by a marked increase in specific mineralocorticoid Steroid ReceptorNA synthesis. These results demonstrate that the interaction of HIV-1 with cell membranes is able to trigger a signal which induces a nuclear infix of 70 kDa heat shock protein. Other studies (J Virological Methods, 26 (1989) 313-318, Rapid detection of HIV-1 in clinical samples by co-culture with heat-shocked cells. M. C. Re, G. Furlini and M. La Placa) demonstrated that mild heat shock allows the detection of intracellular viral markers (p24 core antigen and reverse transcriptase) in a short time compared to controls, showing enhanced infectivity.

In another embodiment, the formulation further includes cDNA clone directed to anti-sense mineralocorticoid Steroid ReceptorNA to heat shock protein. In one embodiment the cDNA clone is directed to the anti-sense mineralocorticoid Steroid ReceptorNA 70 kDa heat shock protein, hsp70. In one embodiment the anti-sense mineralocorticoid Steroid ReceptorNA is directed to the anti-sense mineralocorticoid Steroid ReceptorNA 90 kDa heat shock protein, hsp90.

In another publication (Blochem J. (1995) 311, 797-804, Distant functions of the 90 kDa heat-shock protein (hsp90) in oestrogen and mineralocorticoid receptor activity, effects of hsp90 deletion and mutants, N Binart, M lombes abd E-E Baukiieu), it was demonstrated that when a specific region of hsp90 is mutated that it no longer interacts with the Mineralocorticoid Steroid Receptor.

In another embodiment the anti-sense mineralocorticoid Steroid ReceptorNA and the DNA are delivered to viral infected cells by incorporating the anti-sense mineralocorticoid Steroid ReceptorNA and the DNA into liposomes or carbohydrate vehicles targeted to infected viral cells by placing viral directed antibodies on the surface of these liposomes or vesicles.

In another embodiment of the invention, liposomes are provided carrying the the anti-sense mineralocorticoid Steroid ReceptorNA and the DNA targeted to HIV infected cells by putting antibodies to the HIV coat protein gp160 or gp41 on its surface. The advantage of this is that the liposome can selectively target HIV infected cells.

The outer coat of a lipid envelope virus is constructed of several types of polypeptide chains often arranged in several layers. In many viruses, moreover, the protein capsid is further enclosed by a lipid bilayer membrane the contains proteins. Many of these enveloped proteins acquire this envelope in the process of budding from the plasma membrane. This budding process allows the virus particles to leave the cell without disrupting the plasma membrane and therefore not killing the cell. However, the coat protein of the lipid envelope-virus is significantly different in phospholipid profile than that of the plasma membranes of the host cell. It is thought that this is due to selective sequestration of lipids occurring through the budding process, in which the viral proteins select specific domains within the host cell membrane through which to emerge during maturation. Aldosterone, is one hormone that is selected and sequestered during the budding process. It would seem that a high cholesterol/phospholipid ratio within viral envelopes is required for infectivity of many enveloped viruses.

Examples of viruses include, but are not limited to, human T-cell leukemia virus (HTLV-I), which is indigenous to the Caribbean and to Israel, and human T-cell Leukemia virus III (HTLV-III), which causes hairy cell leukemia.

Further, the invention provides use of the composition in the manufacture of a medicament for use in the prophylaxis or therapy of a viral infection or a complication or consequence thereof.

Additionally, the invention provides use of the composition to provide protection against virus infections in immunocompromised animals and humans. These compositions may be used prophylactically or therapeutically to protect animals or patients from the consequences of infection by pathogenic viruses.

Further, the invention provides use of the composition in veterinary medicine, prophylactically and therapeutically in animal populations that are subject to viral infection that compromises immune response and cause infection.

Additionally, the invention provides use of the composition in the treatment of Immunocompromised AIDS patients or those infected with a retrovirus such as HIV virus showing the AIDS related complex (ARC).

Furthermore, the invention provides a method of rendering a virus non-infectious, but viable for immune clearance and immune memory cell development comprising deleting from its genome the code directed to the Hydrophobic amino acid sequences that bind the virus to the aldosterone binding site on the Mineralocorticoid Steroid Receptor.

The term “antibodies” is defined herein to include not only whole antibodies but also biologically active fragments of antibodies, specifically fragments that contain antigen binding regions. Such antibodies can be prepared by conventional methodology and/or by genetic engineering. Antibody fragments may be genetically engineered, preferably from the variable regions of the light and/or heavy chains (VH and VL), including the hypervariable regions and still more preferably from both the VH and VL regions. For example, the term “antibodies” as used herein comprehends polyclonal and monoclonal antibodies and biologically active fragments thereof including among other possibilities “univalent” antibodies [Glennie et al. Nature, 295:712 (1982); Fab proteins including Fab′ and F(ab)2 fragments whether covalently or non-covalently aggregated; light or heavy chains alone, preferably variable heavy and light chain regions (VH and VL regions) and more preferably including the hypervariable regions [otherwise known as the complimentary regions of said VH and VL regions]; Fc proteins; “hybrid” antibodies capable of binding more than one antigen; constant-variable region chimeras; “composite” immunoglobulins with heavy and light chains of different origins; “altered” antibodies with improved specificity and other characteristics as prepared by standard recombinant techniques and also by oligonucleotide-directed mutagenesis techniques [Dalbadie-McFarland et al., PNAS USA, 79:6409 (1982).

The term idiotypic antibody is an antibody raised against the antigen binding site of another antibody. The antibody is produced using the anti-idiotypic method. For example the monoclonal idiotypic antibody is anti-aldosterone antibody was raised against the aldosterone binding site of the anti-aldosterone antibody.

In general the term steroid is considered as a group name for lipids that contain a hydrogenated cyclopentanoperhydrophenanthrene ring system. The substances of particular interest to the present invention are the adrenocortical hormones, particularly aldosterone.

The term steroid hormone receptors are defined herein as nuclear receptors that are phosphoproteins that include receptors for mineralocorticoids, capeable of binding to its DNA responsive element.

### EXAMPLES

Without further elaboration, it is believed that one skilled in the art can, using the preceding description, practice and present invention to its fullest extent. The following detailed examples describe how to test the various compounds of this invention and/or perform the various processes of the invention and are to be construed as merely illustrative, and not limitations of the preceding disclosure in any way whatsoever. Those skilled in the art will promptly recognize appropriate variations from the procedures. These and other features and advantages of the present invention will be more clearly understood with reference to the following description of some embodiments thereof and with reference to the accompanying drawings in which:

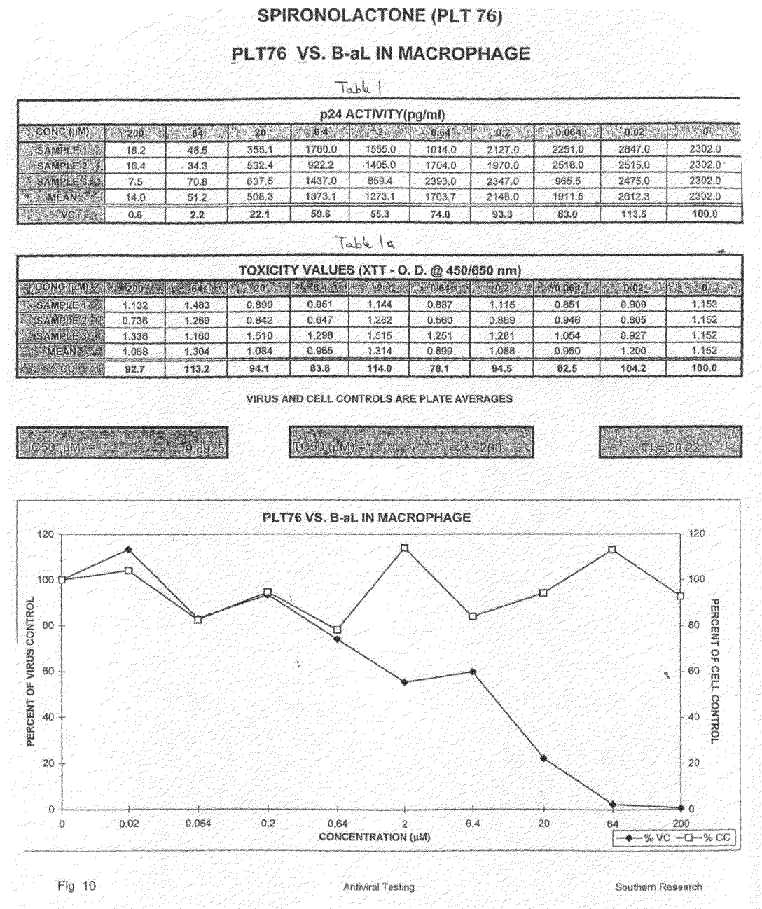

FIG. 10 is a graph showing the effect of spironolactone on HIV-1 IIIB virus (% VC) in macrophages and its effects on cell viability (% CC), while table 1 and 1A depict the individual data shown in FIG. 10;

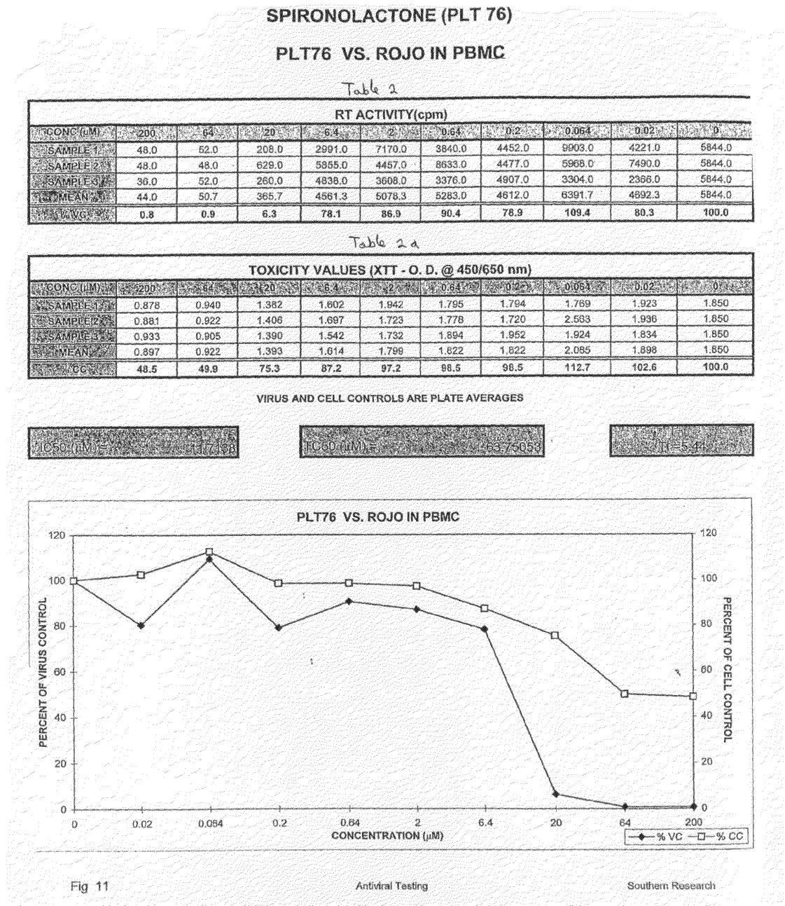

FIG. 11 and table 2 and 2A are another embodiment of the invention, showing the effect of spironolactone on HIV-1 IIB virus in PBMC cells;

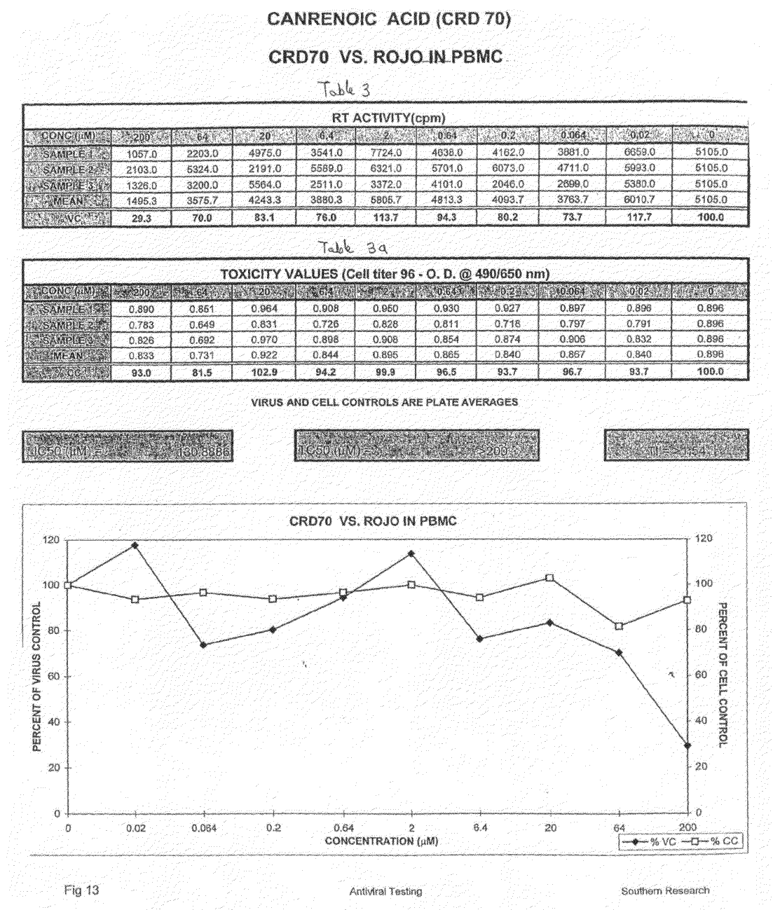

FIG. 13 is another embodiment of the invention showing the effect of the potassium salt of canrenoate (potassium canrenoate) on HIV-1 IIIB virus (% VC) and cell viability in PBMC cells (% CC); and

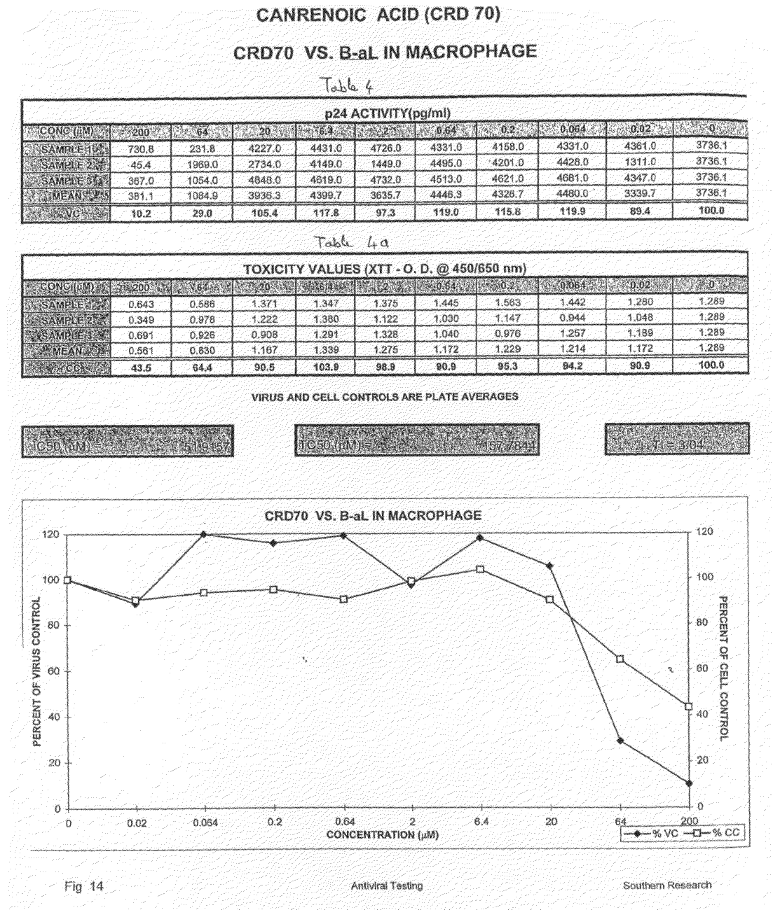

FIG. 14 and table 4 and 4A show the effect of the potassium salt of canrenoate (potassium canrenoate) on HIV IIIB virus in macrophages.

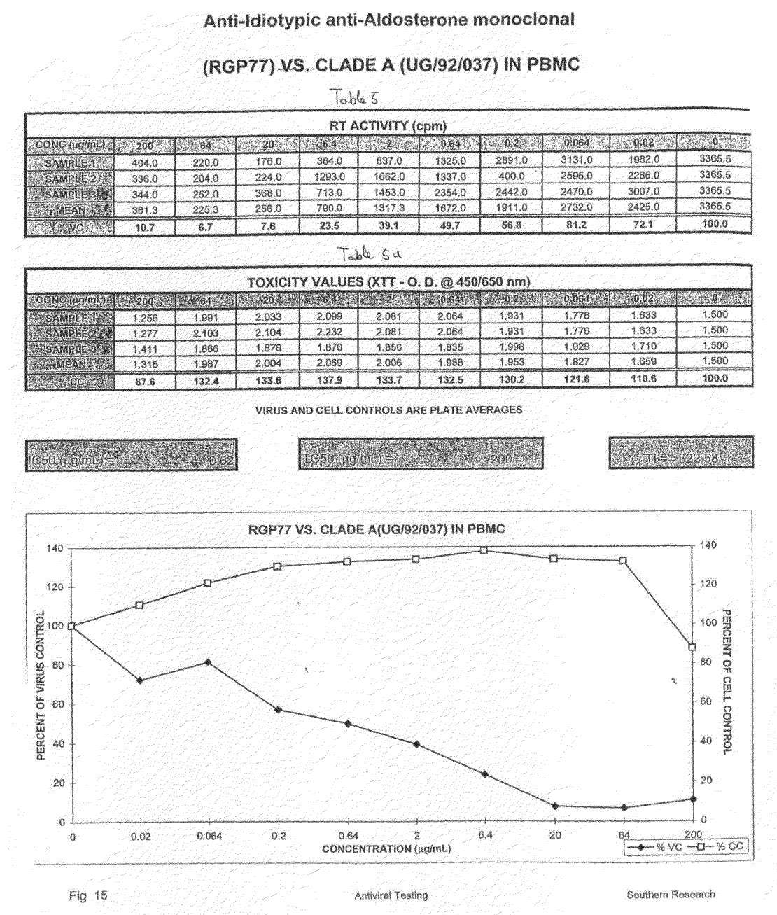

FIG. 15 is a graph showing the effect of anti-idiotypic anti-aldosterone monoclonal antibody on HIV-1 IIB virus (% VC) in peripheral blood mononuclear cells (PBMC) and its effects on cell viability (% CC), while table 5 and 5A depict the individual data shown in FIG. 15;

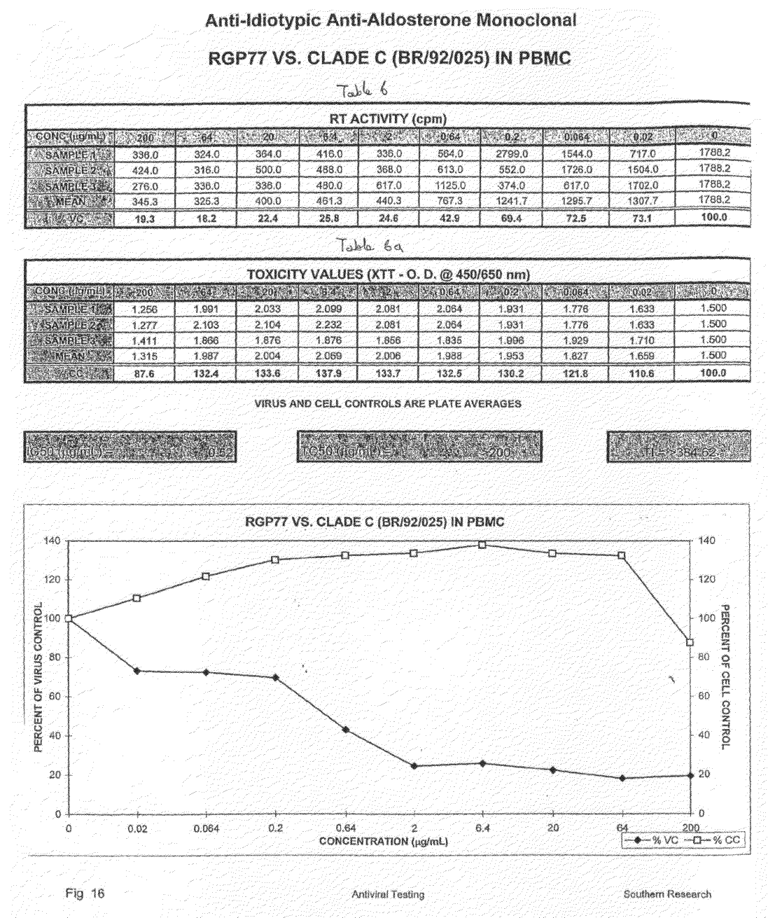

FIG. 16 and table 6 and 6A are another embodiment of the invention, showing the effect of anti-idiotypic anti-aldosterone monoclonal antibody on HIV-1 IIB virus 5 in PBMC cells;

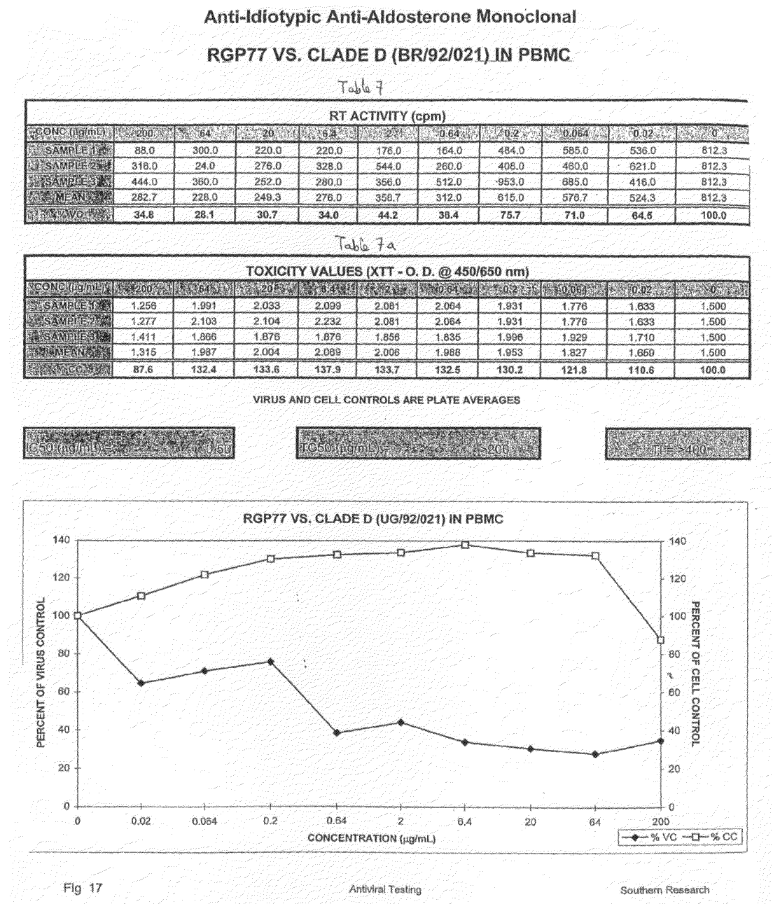

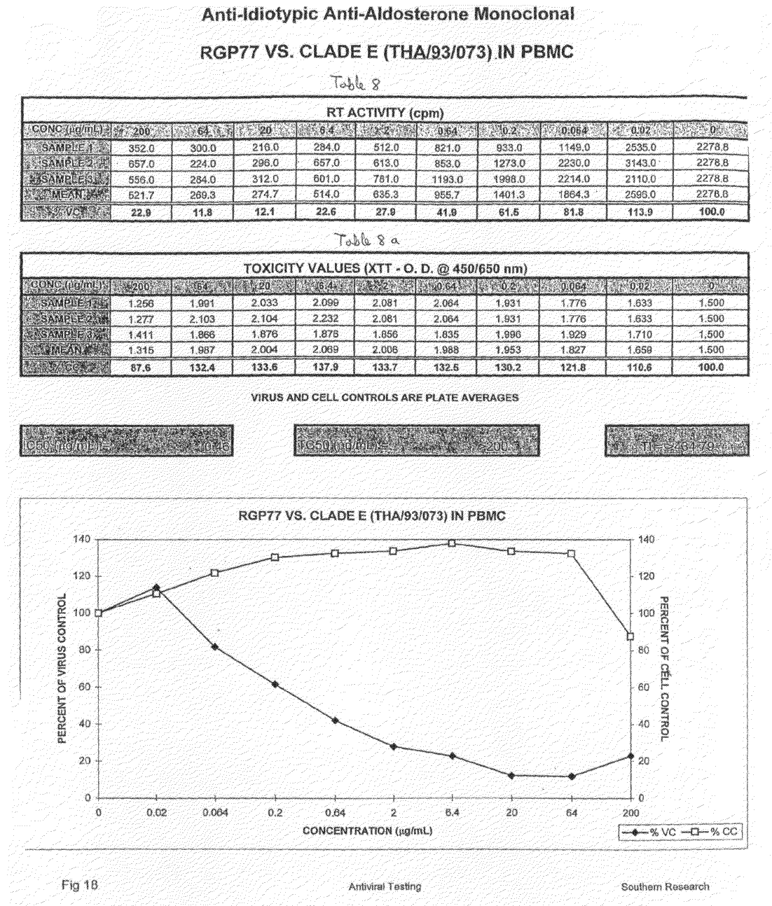

FIG. 17 and tables 7 and 7A and FIG. 18 and tables 8 and 8A are further embodiments of the invention showing the effect of anti-idiotypic anti-aldosterone monoclonal antibody on HIV-1 IIIB in PBMC cells.

### Examples

Examples 1 and 2 demonstrate the method and results of tests that are performed to determine the effectiveness of spironolactone and the potassium salt of canrenoate (Canrenoic acid), the primary metabolite of spironolactone, anti-idiotypic anti-aldosterone monoclonal antibody and aldosterone at reducing the ability of HIV-1 regardless of strain type or clade to infect T-lymphocytes and macrophages. Tests are also carried out to determine the toxicological effect of spironolactone on uninfected T-lymphocytes and macrophages. Examples 3 to 8 describe methods of how the Antimineralocorticoid compounds are formulated, so that upon administration, by several routes of administration, there is a maximum bioavailability.

### Example 1

Anti-HIV Activity in Fresh Human Cells: Assay in Fresh Human T-Lymphocytes

Fresh human peripheral blood lymphocytes (PBL) are isolated from voluntary Red Cross donors, seronegative for HIV and HBV. Leukophoresed blood is diluted 1:1 with Dulbecco's phosphate buffered saline (PBS) and layered over 14 mL of Ficoll-Hypaque density gradient in a 50mi centrifuge tube. Tubes are then centrifuged for 30 minutes at 600 Xg. Banded PBLs are gently aspirated from the resulting interface and subsequently washed 2× with PBS by low speed centrifugation. After final wash, cells are enumerated by trypan blue exclusion are re-suspended at 1×107/ml in RPMI 1640 with 15% fetal bovine serum (FBS), 2 mM L-glutamine, 4 ug/ml PHA-P and allowed to incubate for 48-72 hours at 37″C. After incubation, PBLs are centrifuged and reset in RPMI 1640 with 15% FBS, 2 mM L-glutamine, 100 U/ml penicillin, 100 ug/ml streptomycin, 10 ug/ml gentamycin, and 20 U/ml recombinant human IL-2. PBLs are maintained in this medium at a concentration. of 1-2×10⁶/ml with biweekly medium changes, until use in assay protocol.

For the PBL assay, PHA-P stimulated -cells from at least two normal donors are pooled, set in fresh medium at 2×10⁶/mL and plated in the interior wells of a 96 well round bottom microplate at 50 μL/well. Test drug dilutions are prepared at a 2× concentration in microtiter tubes and 100 μL of each concentration is placed in appropriate wells in a standard format. Fifty microliters of a predetermined dilution of virus stock is placed in each test well. Wells with cells and virus alone are used for virus control. Separate plates are identically set without virus for drug cytotoxicity studies using an XTT assay system.

In the standard PBL assay (MOI: 0.2), the assay is ended on day 7 following collection of cell free supernatant samples for reverse transcriptase activity assay. Tritiated thymidine triphosphate (NEN) (TTP) was resuspended in distilled H₂O at 5 Cl/ml. Poly rA and oligo dT are prepared as a stock solution, which is kept at −20° C. The RT reaction buffer is prepared fresh on a daily basis and consists of 125 μl 1 M EGTA, 125 μl dH₂O, 110 μl 10% SDS, 50 μl 1M Tris (pH 7.4), 50 μl 1M DTT, and 40 μl 1M MgCl₂. These three solutions are mixed together in a ratio of 2 parts TTP, 1 part poly rA:oligo dT, and 1 part reaction buffer. Ten microliters of this reaction mixture is placed in a round bottom microtiter plate and 15 μl of virus containing supernatant was added and mixed. The plate is incubated at 37° C. in a water bath with a solid support to prevent submersion of the plate and incubated for 60 minutes. Following reaction, the reaction volume is spotted onto DE81 filter mats, washed 6 times for 5 minutes each in a 5% sodium phosphate buffer, 2 times for 1 minute each in distilled water, 2 times for 1 minute each in 70% ethanol, and then dried. Opti-Fluor O is added to each filter mat, and incorporated radioactivity is quantitated utilizing a Wallac 1450 Microbetaplus liquid scintillation counter. Toxicity plates are stained with XTT as described above.

The results are presented In FIG. 10 and 11 and Table 1A and Table 1B. Referring to FIG. 10 of the drawings, it can be observed that T-lymphocyte cultures remain healthy at all concentrations of Spironolactone used, while % HIV-1 levels decreased in a dose dependent manner.

Referring to FIG. 13 and 14 of the drawings and Table 3 and 3A, it can be observed that T-lymphocyte cultures similarly remain healthy at all concentrations of potassium canrenoate, while % HIV-1 levels decreased in a dose dependent manner.

Referring to FIG. 15, 16, 17 and 18 of the drawings and Table 5 and 5A. The anti-idiotypic anti-aldosterone monoclonal antibody is a monoclonal antibody that is directed to the bindng site for aldosterone on an anti-aldosterone monoclonal antibody. It competitively inhibits the binding of aldosterone to the Mineralocorticoid Steroid Receptor in a dose dependent fashion. The ant!-idiotypic antibody is defined as a restricted internal image of aldosterone. Moreover it interacts with the receptors specific for-aldosterane and is a very specific Antimineralocorticoid compound. It can be observed that anti-idiotypic anti-aldosterone monoclonal antibody dose dependently inhibits % HIV-1 viral replication in T-lymphocyte cultures. A dose dependent decrease in cell growth was also observed. It is hypothesised that in the absence of aldosterone, that perhaps cellular salt content is affected in vitro which is necessary for life.

### Example 2

Anti-HIV Activity in fresh Human Cells: Assay in Fresh Human monocyte-macrophages

For isolation of adherent cells, 3×10⁶ non-PHA stimulated peripheral blood cells are resuspended in Hanks buffered saline (with calcium and magnesium) supplemented with 10% human AB serum. The cells are placed in a 96-well microtiter plate at 37° C. for 2 hours. Non-adherent cells are removed by vigorously washing six times. The adherent cells are cultured for 7 days in RPMI 1640 tissue culture medium with 15% fetal bovine serum. The cultures are carefully monitored for confluency during this incubation period. Infection of the cells is performed with monocytotropic HIV-1 isolates. High titer pools of each of these viruses are harvested from Infected cultures of peripheral blood adherent cells and frozen in 1.0 ml aliquots at 80° C. Monocyte-macrophage monolayers are infected at an MOI of 0.1. Compounds to be evaluated in the monocyte-macrophage assay are added to the monolayers shortly before infection in order to maximize the potential for identifying active compounds.

At 2 days post-infection, the medium is decanted and the cultures washed twice with complete medium in order to remove excess virus. Fresh medium alone or medium containing the appropriate concentrations of drugs is added and incubation continued for an additional 5 days. XTT staining for cytotoxicity and HIV p24 ELISA assays for production of p24 core antigen are performed on Day 7 post-infection. ELISA kits are purchased from Coulter. The assay is performed according to the manufacturer's recommendations. Control curves are generated in each assay to accurately quantitate the amount of capsid protein in each sample. Data is obtained by spectrophotometric analysis at 450 nm using a Molecular Devices Vmax plate reader. P24 concentrations are calculated from the optical density values by use of the Molecular Device software package Soft Max.

The results are presented in FIG. 13 and 14 and Table 2. The results in macrophages were very similar to those observed in the T-lymphocytes. HIV-1 levels decreased in a dose dependent fashion for both drugs with little effect on cell viability.

The results in macrophages were very similar to those observed in the T-lymphocytes for potassium canrenoate. HIV-1 levels decreased in a dose dependent fashion for both drugs with little effect on cell viability.

Referring to FIG. 15, 16, 17 and 18 and tables 6 and 6A. HIV-1 levels decreased in a dose dependent fashion for the ant!idiotypic anti-aldosterone monoclonal antibody with little effect on cell viability.

### Example 3

This example describes how an Antimineralocorticoid compound can be prepared with cyclodextrin according to the present invention.

Preparation of Spironolactone in Hydroxypropyl-Beta-cyclodextrin (HP-beta-CD) Solution

1. Forty five grams of HP-beta-CD Is weighed out on an analytical balance, and placed into a clean beaker.

2. To this one litre of sterile physiological saline is added.

3. The resultant mixture is covered and mixed on a stirrer for four hours or overnight, until a clear solution is obtained.

4. Non-micronised spironolactone is added to this, at a concentration of 20 mg/ml, and is stirred again, until the solution is clear.

5. This is then passed through a 0.2 micron filter to sterilise it, and is then aliquoted into sterile containers.

### Example 4

The example describes one method of encapsulating an Antimineralocorticoid compound by enterically coating with a cellulose acetate phthalate film. The use of enteric coating targets the release of the composition in the lower gastrointestinal tract The manufacture of enterically coated capsules, cachets, powders, tablets etc, is well known in the art. The following is provided by way of example:

Cellulose acetate phthalate films

| | % Parts by weight in |
|---|---|
| Constituents | finished formulation |
| Cellulose acetate phthalate | 5.56 |
| Diethyl phthalate | 3.34 |
| (60% based on the film former) | |
| Composition | 22.78 |
| Methylene chloride | |

Solid amount of spray suspension: 8.9%
Content of dry polymer substance: 5.56%
Amount applied: 32.5 mg film/capsule

The composition can include at least one Antimineralocorticoid compound. This is a preferred embodiment of the invention.

### Example 5

An alternative method to formulate the compositions is put them into liposomes of appropriate size. The manufacture of liposomes and the insertion of active ingredients into such liposomes is well known in the art. The following is provided by way of example.

Preparation of Spironolactone in Liposomes.

1. 400 mg phosphatidyl choline, 100 mg cholesterol and 80 mg of spirolactone are dissolved in a solution of chloroform:methanol (2:1, v/v).

2. They mixture is then dried by rotary evaporation onto the sides of a round bottomed flask.

3. The resultant film is rehydrated by adding 8 mls of 0.9% w/v sodium chloride solution.

4. The size of the resultant liposomes are measured by photon correlation spectroscopy using a Malvern Zetasizer 3000.

5. If required, liposomes can be produced below 400 nm by the use of sonication.

Formulations that comprise liposomes can be delivered to a subject by any standard route, e.g., oral, aerosol or parenteral (e.g., i.v. or i.m.).

### Example 6

By formulating the Antimineralocorticoid compounds in a suppository formulation, they can be administered rectally. Formulations for rectal administration may be presented as a suppository with a suitable base comprising for example cocoa butter or a salicyclate. The following is included by way of example:

Preparation of Spironolactone in Suppository Form.

1. Spironolactone is weighed out accurately, the amount dependant on how many suppositories will be produced. There will be 500 mg per suppository.

2. The spironolactone is blended with the suppository base, forming an homogenous mixture of the two.

3. The mixture is poured into individual plastic casings, which are sterilely sealed, and left to harden.

The suppository base can be a series of triglycerides derived from edible vegetable oils, which may resemble cocoa butter in their properties. They are extremely stable, uniform in condition, which results in precise mewing characteristics and need no special storage conditions. Typical properties may be:

| | |
|---|---|
| FFA %: | 0.1 |
| Saponification value: | 242 |
| Iodine value: | 3 |
| Moisture %: | 0.1 |
| Melting Point, | 35° C. |
| closed capillary | |

### Example 7

Preparation of Spironolactone for IV Administration by Attaching to Polyethylene glycol.

Another method of administering the Antimineralocorticoid compounds, is to administer them parenterally. One method of achieving this is by attaching them to a carrier molecule, for example a biocompatible polymer. One of the best biocompatible polymers is polyethylene glycol (PEG) due to its many useful properties including: lack of toxicity and immunogenicity, nonbiodegradability and ease of excretion from living organisms, increased circulating half-life in blood, potentially increasing bioavailability and potency.

The term PEG means an ethylene glycol polymer that contains about 20 to about 2000000 linked monomers, typically about 50-1000 linked monomers, usually about 100-300. Polyethylene glycols include PEGs containing various numbers of linked monomers e.g. PEG20, PEG30, PEG40, PEG60, PEG80, PEG100, PEG115, PEG200, PEG300, PEG400, PEG500, PEG600, PEG1000, PEG1500, PEG2000, PEG3350, PEG4000, PEG4600, PEG5000, PEG6000, PEG8000, PEG11000, PEG12000, PEG2000000 and any mixtures thereof.

Alternatively, the compounds can be mixed with an excipient. The term excipient means a component or an ingredient that is acceptable in the sense of being compatible with the other ingredients of invention compositions or formulations and not overly deleterious to the patient or animal to which the formulation is to be administered. As used herein, excipients are usually liquids, including benzyl benzoate, cottonseed oil, N,N-dimethylacetamide, an alcohol such as a C₂₋₁₂ alcohol (e.g. ethanol), glycerol, peanut oil, PEG, vitamin E, poppyseed oil, propylene glycol, safflower oil, sesame oil, soybean oil and vegetable oil. Excipients as used herein will usually exclude chloroform, dioxane and DMSO. Excipients comprise one or more components typically used in the pharmaceutical formulation arts, e.g. fillers, binders, disintegrants and lubricants.

Inventions compositions suitable for parenteral delivery of Antimineralocorticoid compounds to humans or animals typically comprise two or three or more excipients. Exemplary embodiments include (1) any two, three or four of propylene glycol, PEG200, PEG300, ethanol and benzyl benzoate and (2) any two, three or four of propylene glycol, PEG100, PEG200, PEG300, PEG400 and benzyl benzoate.

### Example 8

Additional Formulation of Spironolactone

1. To a glass vessel add 400 mg of spironolactone and 2.5 ml of polyethylene glycol 300 and vortex for 1 minute to form a smooth, creamy liquid.

2. Add 2.5 ml of propylene glycol and vortex for 1 minute to form a uniform suspension.

3. Add 0.5 ml of benzyl benzoate and vortex for 1 minute to form a semi-clear liquid.

4. Add 1.25 ml of absolute ethanol (99.5%) and vortex to a clear and colourless solution.

5. Make up 10 ml with propylene glycol.

6. Store at 10-250° C., avoiding heating of any kind.

This formulation is administered into the subcutaneous layer of the skin.

The term “lower gastrointestinal tract” means here the lower part of the small intestine (ileum) and the colon. The term “enteric coating” means here a coating surrounding the core, the solubility of the coating being dependent on the pH in such a manner that it prevents the release of the drug in the stomach but permits the release of the drug at some stage after the formulation has emptied from the stomach.

Pharmaceutically acceptable refers to those properties and/or substances, which are acceptable to the patient from a pharmacological/toxicological point of view including bioavailability and patient acceptance or to the manufacturing chemist from a physical-chemical point of view regarding composition, formulation, stability and isolatability.

The terms “comprise, comprised and comprising”, and the terms “include, included and including” are used interchangeably in this specification and are to be afforded the widest interpretation.

The invention is not limited to the embodiments described above, but may be varied in both construction and detail within the scope of the claims.

1. A pharmaceutical composition comprising at least one antimineralocorticoid compound or molecule.

2. The pharmaceutical composition claimed in claim 1, wherein the antimineralocorticoid compound is selected from the group consisting of spironolactone, spirorenone, 1,2-dihydro-spirorenone, 1,2α-methylene-spirorenone, eplerenone, drospirenone, potassium canrenoate, canrenoate, canrenone and pharmaceutically acceptable salts thereof, and their metabolites thereof.

3. The pharmaceutical composition as claimed in claim 2, wherein the antimineralocorticoid compound is spironolactone.

4. The pharmaceutical composition as claimed in claim 1 wherein the antimineralocorticoid compound is an anti-idiotypic monoclonal antibody which has binding specificity to a binding epitope present on aldosterone.

5. The pharmaceutical composition as claimed in claim 2 wherein the antimineralocorticoid compound is drospirenone.

6. The pharmaceutical composition as claimed in claim 1 wherein the antimineralocorticoid compound is selected from the group consisting of progesterone, gestodene, dimethisterone, drospirenone, ethinyloestradiol, ethisterone, 11β-hydroxyprogesterone, 17α-hydroxyprogesterone, 16α-methyl progesterone, hydroxyprogesterone caproate, medroxyprogesterone acetate, proligestone and pharmaceutically acceptable salts thereof, and metabolites, thereof.

7. The pharmaceutical composition as claimed in claim 1 wherein, the antimineralocorticoid compound is a 7α-acetylthio-4-pregnene-3,20-dione represented by formula B 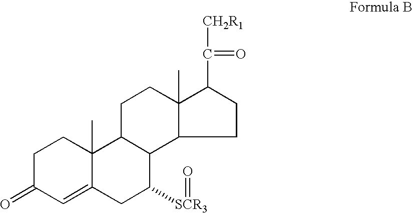
in which R₁ is selected from the group consisting of hydrogen, hydroxy, hydroxyl, a mineral acid ester and acyloxy-OR₂, wherein the acyl group R₂ is derived from a carboxylic acid of the formula R₄OOH which comprises up to 12 carbon atoms and in which R₄ is substituted or unsubstituted, saturated or unsaturated, straight chain or branched, alicyclic, aryl, heterocyclic or mixed, And R₃ is methyl.

8. The pharmaceutical composition as claimed in claim 7 wherein, R₁ is hydroxyl or OR₂, and R₂ is derived from a carboxylic acid from 3 to 12 carbon atoms.

9. The pharmaceutical composition as claimed in claim 7 wherein R₁ is selected from the group consisting of hydroxy, monocarboxylic and a straight or branched-chain alkanooyloxy group having up to 12 carbon atoms.

10. The pharmaceutical composition formulation as claimed in claim 7 wherein R₁ is selected from the group consisting of hydrogen, hydroxy, acetoxy, propionyloxy, n-butyryloxy, trimethylacetoxy, n-valeroyloxy and n-heptanoyloxy.

11. The pharmaceutical composition as claimed in claim 7 wherein the antimineralocorticoid compound is selected from the group consisting of:
7α-acetylthio-4-pregnene-3,20-dione;
7α-acetylthio-21-hydroxy-4-pregnene-3,20-dione;
7α-acetylthio-21-acetoxy-4-pregnene-3,20-dione;
7α-acetylthio-21-propionyloxy-4-pregnene-3,20-dione;
7α-acetylthio-21-n-butyryloxy-4-pregnene-3,20-dione;
7α-acetylthio-21-trimethylacetoxy-4-pregnene-3,20-dione;
7α-acetylthio-21-n-valeroyloxy-4-pregnene-3,20-dione;
7α-acetylthio-21-heptanoyloxy-4-pregnene-3,20-dione;
7α-acetylthio-3-oxo-4,15-androstadiene-[17(β-1′)-spiro-5′]perhydrofuran-2′-one;
3-oxo-7α-propionylthio-4,15-androstadiene-[17(β-1′)-spiro-5′]perhydrofuran-2′-one;
6β,7β-Methylene-3-oxo-4,15-androstadiene-[17(β-1′)-spiro-5′]perhydrofuran-2′-one;
15α, 16α-Methylene-3-oxo-7α-propionylthio-4-androstene-[17(β-1′)-spiro-5′]perhydrofuran-2′-one;
6β,7β,15α, 16α-Dimethylene-3-oxo-4-androstene-[17(β-1′)-spiro-5′]perhydrofuran-2′-one;
7α-Acetylthio-15α,16α-methylene-3-oxo-4-androstene-[17(β-1′)-spiro-5′]perhydrofuran-2′-one;
7α-Acetylthio-15β, 16β-methylene-3-oxo-4-androstene-[17(β-1′)-spiro-5′]perhydrofuran-2′-one;
15β, 16β-Methylene-3-oxo-7β-propionylthio-4-androstene-[17(β-1′)-spiro-5′]perhydrofuran-2′-one; and
6β,7β,15β,16β-Dimethylene-3-oxo-4-androstene-[17(β-1′)- spiro-5′]perhydrofuran-2′-one.

12. The pharmaceutical composition claimed in claim 1 wherein the antimineralocorticoid compound is a 9,11-epoxy steroid compound.

13. The pharmaceutical composition claimed in claim 1, wherein the antimineralocorticoid compound is halogenated.

14. The pharmaceutical composition as claimed in claim 13, wherein the halogen is selected from the group consisting of chlorine, bromine, fluorine and iodine.

15. The pharmaceutical composition claimed in claim 1, wherein the composition further includes at least one other anti-viral agent.

16. The pharmaceutical composition as claimed in claim 15, wherein the anti-viral agents are selected from the group consisting of nucleoside analogues; non-nucleoside reverse transcriptase inhibitors; protease inhibitors; ALX40-4C; hydroxyurea; lobucavir; pentafuside; T-1249; PRO 542; FP-21399; AMD 3100; HE-2000 and peptide T.

17. The pharmaceutical composition as claimed in claim 15, wherein the antiviral agents are selected from the group consisting of Abacavir; Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Coviracil; Cytarabine Hydrochloride; Delavirdine Mesylate; Desciclovir; Didanosine; Disoxaril; Edoxudine; Emivirine; Emtricitabine; Enviradene; Enviroxime; Epivir; Famciclovir; Famotine Hydrochloride; Fiacitabine; Fialuridine; Fosarilate; Foscamet Sodium; Fosfonet Sodium; Ganciclovir; Ganciclovir Sodium; Idoxuridine; Indinavir; Kethoxal; Lamivudine; Lobucavir; Lodenosine; Lopinavir, Memotine Hydrochloride; Methisazone; Nelfinavir; Nevirapine; Penciclovir; Pirodavir; Ribavirin; Rimantadine Hydrochloride; Saquinavir Mesylate; Ritonavir; Somantadine Hydrochloride; Sorivudine; Statolon; Stavudine; Tenofovir; Tilorone Hydrochloride; Trifluridine; Valacyclovir Hydrochloride; Vidarabine; Vidarabine Phosphate; Vidarabine Sodium Phosphate; Tipranavir, Viroxime; Zalcitabine; Zidovudine and Zinviroxime.

18. The pharmaceutical composition as claimed in claim 1, wherein the composition further comprises a protease inhibitor.

19. The pharmaceutical composition as claimed in claim 1, wherein the composition further comprises a pharmaceutically acceptable carrier.

20-23. (canceled)

24. The pharmaceutical composition as claimed in claim 1 wherein the composition has an enteric coating made of a polymer.

25. The pharmaceutical composition as claimed in claim 24 wherein the enteric coating consists essentially of a polymer or copolymer selected from the group consisting of poly(lactic-glycolic acid) polyester, cellulose acetate phthalate, hydroxypropyl-methyl cellulose phthalate poly(butyl methacrylate), (2-dimethyl aminoethyl) methacrylate and methyl methacrylate.

26. (canceled)

27. The pharmaceutical composition as claimed in claim 1, wherein the composition is formulated into liposomes or carbohydrate or cyclodextrin vehicles.

28. The pharmceutical composition as claimed in claim 27, wherein the liposomes or carbohydrate vehicles are targeted to HIV infected cells by conjoining antibodies with a binding specificity for viral-specific binding epitopes to their surface.

29. The pharmaceutical composition as claimed in claim 28, wherein the viral antibodies are directed to at least one of the HIV coat protein gp160, gp120 and gp41.

30. (canceled)

31. The pharmaceutical composition as claimed in claim 1, wherein the composition is a unit dose that comprises 5-500 mg of the antimineralocorticoid compound, alone or in combination with other antivirals.

32. (canceled)

33. The pharmaceutical composition as claimed in claim 1, wherein said antimineralocorticoid compound is a pro-drug entity.

34-65. (canceled)

66. A method of preparing a vaccine comprising a virus, the method comprising the step of mutating or deleting from the viral genome nucleotides which encode for hydrophobic amino acid residues which cause binding of the virus to an aldosterone binding site on a mineralocorticoid steroid receptor, wherein the deletion or mutation prevents expression of the hydrophobic amino acid residues by the virus.

67. A method for the treatment and/or prophylaxis of a viral infection in a subject, the method comprising the step of:
administering a therapeutically effective amount of a pharmaceutical composition comprising at least one antimineralocorticoid compound to a the subject.

68. The method as claimed in claim 67, wherein the viral infection is infection by a retrovirus.

69. The method as claimed in claim 68, wherein the retrovirus is selected from the group consisting of HIV, Herpes virus and cytomegalovirus.

70. (canceled)

71. (canceled)

72. (canceled)

73. The method as claimed in claim 67 wherein the antimineralocorticoid compound is selected from the group consisting of spironolactone, spirorenone, 1,2-dihydro-spirorenone, 1,2α-methylene-spirorenone, eplerenone, drospirenone, potassium canrenoate, canrenoate and canrenone, and pharmaceutically acceptable salts and metabolites thereof.

74. The method as claimed in claim 67 wherein the antimineralocorticoid compound is spironolactone or a pro-drug, derivative or analogue thereof and the viral infection is HIV.

75. The method as claimed in claim 67 wherein the antimineralocorticoid compound is an anti-idiotypic monoclonal antibody which has binding specificity to a binding epitope present on aldosterone.

76. The method as claimed in claim 67, wherein the antimineralocorticoid compound is selected from the group consisting of progesterone, gestodene, dimethisterone, ethinyloestradiol, ethisterone, 11β-hydroxyprogesterone, 17α-hydroxyprogesterone, 16α-methyl progesterone, hydroxyprogesterone caproate, medroxyprogesterone acetate and proligestone, and pharmaceutically acceptable salts and metabolites thereof.

77. The method as claimed in claim 67, wherein, the antimineralocorticoid compound is a 7α-acetylthio-4-pregnene-3,20-dione represented by Formula B: 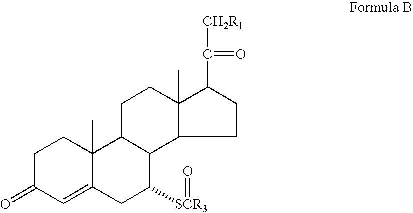
in which R₁ is selected from the group consisting of hydrogen, hydroxy, hydroxyl, a mineral acid ester, a nitrate group and acyloxy-OR₂, wherein the acyl group R₂ is derived from a carboxylic acid of the formula R₄OOH which comprises up to 12 carbon atoms and in which R₄ is substituted or unsubstituted, saturated or unsaturated, straight chain or branched, alicyclic, aryl, heterocyclic or mixed, and wherein R₃ is methyl.

78. The method as claimed in claim 67, wherein the composition further comprises at least one further anti-viral agent.

79. The method as claimed in claim 67, wherein the composition further comprises a protease inhibitor.

80. The method as claimed in claim 67, wherein the viral infection is infection by a virus selected from the group consisting of a retrovirus, a togavirus, a flavivirus, a rubivirus, a pestivirus, a lipid envelope virus, a picornavirus, a rhinovirus, a coronavirus, a respiratory syncytial virus, a poliovirus, a parainfluenza virus, influenza virus, hantavirus, HIV, HTLV-1, HTLV-3, Kaposi's sarcoma-associated herpes virus, HHV-6, HHV-8, the viruses of the genus Molluscipoxvirus, HAV, HBV, HCV, Epstein Barr virus, Herpes virus and cytomegalovirus.

81. The method as claimed in claim 67, wherein said subject is a neonate and the pharmaceutical composition is administered prior to delivery of said neonate and/or during delivery of said neonate.

82. The method as claimed in claim 67, wherein the composition is administered enterally, parenterally, topically, orally, rectally, nasally or vaginally.

83. The method as claimed in claim 67, wherein the composition is administered intermittently.

84. A method for the treatment and/or prophylaxis of an AIDS related syndrome in a subject, the method comprising the step of:
administering a therapeutically effective amount of a pharmaceutical composition comprising at least one antimineralocorticoid compound to the subject.

85. The method of claim 84 wherein the AIDS related syndrome is selected from the group consisting of cachexia, wasting syndrome, lipodystrophy, and combinations thereof.

86. A vaccine for immune clearance and immune memory cell development, the vaccine comprising a viral genome wherein the nucleotides which encode for the hydrophobic amino acid residues, which cause binding of the virus to an aldosterone binding site on a mineralocorticoid steroid receptor, have been deleted or mutated such that expression of the hydrophobic amino acid residues is prevented.

87. The pharmaceutical composition as claimed in claim 16, wherein:
the nucleoside analogues are selected from the group consisting of AZT, ddC, ddl, d4T, 3TC, BW 1592, PMEA/bis-POM PMEA, dOTC, and DAPD;
the non-nucleoside reverse transcriptase inhibitors are selected from the group consisting of delavirdine, DMP 266, HBY097, loviride, nevirapine, emivirine, AG1549, PNU142721, Calanolide A, and DPC961; and
the protease inhibitors are selected from the group consisting of ABT-378, ritonavir, nelfinavir, BW 141, KNI-272, indinavir, saquinavir, L-756,423, DMP-450 and BMS-232630.


SEQUENCE LISTING (2 sequences)

SEQ ID NO: 1 - DNA, 15 bp, Artificial
  GRE(+) DNA nucleotide sequence coding for the hydrophobic amino acid sequence corresponding to the aldosterone attachment site
ggtacannntgttct

SEQ ID NO: 2 - DNA, 15 bp, Artificial
  GRE(-) DNA nucleotide sequence coding for the hydrophobic amino acid sequence corresponding to the aldosterone attachment site
atyacnnnntgatcw